(19) United States
(12) Patent Application Publication
(10) Pub. No.: US 2017/0367901 A1
(43) Pub. Date: Dec. 28, 2017

(54) ENHANCED SANITARY PAD

(71) Applicant: Deborrah R. Peoples Ellis, Miami, FL (US)

(72) Inventor: Deborrah R. Peoples Ellis, Miami, FL (US)

(21) Appl. No.: 15/635,154

(22) Filed: Jun. 27, 2017

Related U.S. Application Data
(60) Provisional application No. 62/493,233, filed on Jun. 27, 2016.

(51) Int. Cl. A61F 13/42; A61F 13/475; A61F 13/58

(52) U.S. Cl. CPC A61F 13/42; A61F 13/58; A61F 13/475; A61F 2013/421

(57) ABSTRACT

An Enhanced Sanitary Pad has various optional devices associated therewith in order to ameliorate painful conditions and also signal the amount of liquids absorbed therein. In this regard, cooling crystals are embedded within an absorbent layer attached to a plastic backing material. Another option has a cooling strip associated with the pad at either end thereof, on the longitudinal sides of the pad, at a side of an optional raised protrusion located on the pad or on the raised protrusion itself. This protrusion is also formed of tightly wound absorbent material and is either stitched or supported with adhesive interlay to maintain its shape; it is optionally removable from the pad and associated therewith using corresponding velcro interlocks.

### FIELD OF THE INVENTION

The present invention relates to sanitary pads for absorbing feminine discharges such as menstrual fluids and other such liquids. More particularly, the present invention relates to sanitary pads that provide a close fitting pad thereby improving the wearability of the item as well as simultaneously maintaining the integrity of the absorbent materials therein.

### BACKGROUND OF THE INVENTION

### Cooling Materials

Cooling materials exist in many disciplines. For example, U.S. Pat. No. 9,402,772 to Fernkvist and herein incorporated by reference; this patent teaches an absorbent article includes a breathable backsheet material layer. At least a part of the layer includes a polymeric material exhibiting a discontinuous change in free volume in response to any of the following conditions or variables: liquid contact, the presence of certain ions, temperature and/or pH. One example of such polymeric materials is side chain liquid crystalline polymers which are able to undergo a phase transition between an isotropic phase and a nematic phase. The discontinuous change in free volume involves an abrupt change of the permeability of the breathable material layer.

Further in column 7, lines 1-24 the patent continues: The Intelimer® polymer will undergo a discontinuous change of free volume at a temperature which is selected to be slightly above the wet temperature for body liquid, such as urine or blood, i.e. in the temperature range 5-50° C. depending on the environmental temperature. Above this temperature the polymer will have an isotropic (amorphous) structure in which the free volume between the polymer chains is large enough to let air and water vapour pass therethrough. When the absorbent article is wetted by body liquid and the liquid reaches the backsheet material, an evaporation process will start as the body liquid starts to evaporate and the vapours escape through the backsheet.

This will result in a cooling effect, so that the temperature in the area where the backsheet is in contact with the evaporating body liquid will be lowered to the wet temperature for body fluids like urine and blood. When reaching this temperature the polymer will undergo an abrupt discontinuous change in free volume and switch to a nematic (crystalline) phase, in which the free volume between the polymer chains is smaller and thus the permeability of the film is decreased. This will lead to a rapid and effective sealing effect reducing the risk for leakage of liquid through the backsheet. The permeability to air and vapours will of course also be reduced, but may be maintained to a certain degree.

Another type of cooling device is currently sold by Fashion Bed Group (http://fashionbedgroup.com/). This company sells various products containing polymers that it calls Crystal Gels and holds a trademark for Aere™ Crystal™ Gel Mattress & Pillow that come in various sizes. It is through the use of innovative crystal gel fibers, both the mattress and pillow protector provide a more comfortable, cooling surface. These products are woven with the rest of the material such that the crystal gel fibers cool by absorbing heat and dissipating it away from the body.

Additionally, there are various other types of cooling gels on the marketplace such as as a topical gel of methyl salicylate. Further, various over the counter gels having aloe vitamin E and other ingredients currently exist to provide cooling to areas of the body needing this treatment. Thus, there are several ways of cooling fabrics and other fibrous materials using the above innovative materials and other materials not specifically described herein.

### Vaginal Devices

Various vaginal devices exist in the marketplace that are inserted within a lady's private parts so as to absorb vaginal discharge. However, many women do not like to use the vaginal inserts because of various issues. Amongst these are discomfort, hygiene issues, religious reasons, as well as the fact that it has been shown that these devices can have toxic effects because of the various chemicals embedded therein.

### Sanitary Pads

Billions of women throughout the world experience a monthly menstrual discharge. The use of sanitary pads for absorbing feminine menstrual discharge is well known in the prior art. The primary goal of a sanitary pad is to discreetly and completely absorb the menstrual discharge without embarrassing leakage or unsightly bulges that would reveal the aforementioned. Numerous types of sanitary pads exist in the prior art and have been disclosed in various patent documents.

A sanitary pad is made of layer(s) of cellulose extracted from the walls of trees and plants associated at manufacture with layer(s) of super absorbent polymer powder derived from petroleum or other sources; alternatively, the two materials are combined into a single or multiple layer(s) of integral material. The polymer powder can absorb many times its volume and along with the cellulose is sandwiched within one or more layers of plastic backing material. If using a single plastic layer it folds back upon itself trapping the edge of the cellulose/polymer layer(s) within the plastic layer.

However, the most common manufacturing technique is for two outer layers. A first one is perforated to pass menstrual and other liquids therethrough into the absorbent cellulose and polymer; a second one does not have any perforations and attaches at its periphery with the corresponding first one so as to preserve moisture integrity within the absorbent materials sandwiched there between. Of course, adhesives, heat treatment, glues, or other types of chemical treatment keep the absorbent materials attached between the two plastic outer layers.

However, one problem with this system that has not been addressed is that users sometimes experience rashes or fail to recognize when the pad has become full. Thus, there needs to be some way to ameliorate this discomfort and also to trigger recognition of the amount of liquids contained in the absorbent material contained within the confines of the pad.

Another issue with most modern sanitary pads is that they have no way of ensuring a correct fit because of the limitations of their manufacture. In other words, they are fashioned to be near to or adjacent the vaginal labia; however, because of their size and shape as well as daily user activity the pad shifts location which can potentially release liquids to the outer clothing. Other problems of form, fit and finish negatively impact the wearer as one size does not fit all shapes and sizes; on the contrary, pads are currently designed mainly by sizes whilst each person has unique needs.

Further, clothing differences (skirt, pants, shorts, underwear) also impact the friction containment of the pad in the underwear and as a result, the pad never truly forms an adequate liquid resistant seal with the female form. On the contrary, the current sanitary pad structure does not provide maximum leak protection and does not restrict the motion of the pad which can thereby causes potential embarrassing leakage of menstrual or other fluids.

Accordingly, there needs to be some solution to the aforementioned problems that ensures a liquid resistant seal at the feminine vaginal opening as well as restricting motion so as to complement the aforementioned seal.

### SUMMARY OF THE INVENTION

The present invention overcomes the deficiencies of the known art and the problems that remain unsolved by providing an enhanced sanitary pad as described below.

A sanitary pad comprising:
a first plastic layer having
an absorbent material attached thereto and
a protrusion attached to the sanitary pad and extending out therefrom.

In another aspect, further comprising:
cooling crystals embedded within the absorbent material.

In another aspect, further comprising:
a cooling strip attached to the sanitary pad.

In another aspect, wherein the cooling strip is disposed on a side of the protrusion.

In another aspect, wherein the cooling strip is disposed adjacent to a side of the protrusion.

In another aspect, wherein the cooling strip is disposed at an end of the sanitary pad.

In another aspect, wherein the protrusion is removable from the sanitary pad.

In another aspect, wherein the protrusion has a first attachment integrally attached thereto.

In another aspect, wherein the sanitary pad has a corresponding second attachment integrally attached thereto for removal of the protrusion.

A hygiene warning system comprising:
a first plastic sheet having
an absorbent layer attached thereto and
cooling crystals associated with the absorbent layer.

In another aspect, further comprising:
a cooling strip attached to the hygiene warning system.

In another aspect, wherein the cooling strip is disposed on a side of a protrusion associated with the hygiene warning system.

In another aspect, wherein the cooling strip is disposed adjacent to a side of a protrusion associated with the hygiene warning system.

In another aspect, wherein the cooling strip is disposed at an end of the hygiene warning system.

In another aspect, further comprising:
a protrusion associated with the hygiene warning system.

In another aspect, wherein the protrusion is removable from the sanitary pad.

In another aspect, wherein the protrusion has a first attachment integrally attached thereto.

In another aspect, wherein the hygiene warning system has a corresponding second attachment integrally attached thereto for removal of the protrusion.

A liquid absorbing napkin comprising:
a base layer having
an absorbent material integrally attached to the base layer;
   wherein the liquid absorbing napkin has a cooling system associated therewith.

In another aspect, wherein the cooling system is from a group of cooling systems comprising: a cooling strip, cooling crystals.

These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, in which:

FIG. 1A presents an Enhanced Sanitary Pad having cooling crystals embedded within the absorbent material in an embodiment disclosed herein. FIG. 1B presents an Enhanced Sanitary Pad having a raised protrusion in an embodiment disclosed herein. FIG. 1C presents an Enhanced Sanitary Pad having a raised protrusion and cooling crystal embedded within the absorbent material in an embodiment disclosed herein.

FIG. 2A presents an Enhanced Sanitary Pad having cooling crystals and longitudinal cooling strips in an embodiment disclosed herein.

FIG. 2B presents an Enhanced Sanitary Pad having cooling crystals and transverse cooling strips at opposite narrow ends of the pad in an embodiment disclosed herein.

FIG. 2C presents an Enhanced Sanitary Pad having cooling crystals and transverse cooling strips at opposite narrow ends of the pad as well as longitudinal cooling strips in an embodiment disclosed herein.

FIG. 2D presents an Enhanced Sanitary Pad having cooling crystals and transverse cooling strips at opposite narrow ends of the pad as well as longitudinal cooling strips and a raised protrusion in an embodiment disclosed herein.

FIG. 2E presents an Enhanced Sanitary Pad having cooling crystals as well as longitudinal cooling strips disposed on opposite sides of a raised protrusion in an embodiment disclosed herein.

FIG. 2F presents an Enhanced Sanitary Pad having cooling crystals as well as transverse cooling strips disposed on opposite ends of the pad as well as having a raised protrusion in an embodiment disclosed herein.

FIG. 3 presents a plastic strip integral with an Enhanced Sanitary pad having a cooling gel thereon covered by a removable plastic strip used in an Enhanced Sanitary Pad in an embodiment disclosed herein.

FIG. 4 presents a raised protrusion having a cooling strip thereon for use in an Enhanced Sanitary Pad in an embodiment disclosed herein.

FIG. 5 presents removable raised protrusion useable in an Enhanced Sanitary Pad in an embodiment disclosed herein.

Like reference numerals refer to like parts throughout the several views of the drawings.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in each figure.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The pads taught herein are formable in various shapes so as to improve the fit and liquid absorption. Tape and or wings are also additional options that are combinable with any of the embodiments taught herein. These pads have increased absorbability, leak prevention, enhanced comfort and obscurity. Additionally, the pads have a cooling option to alert a user when the pad is almost full.

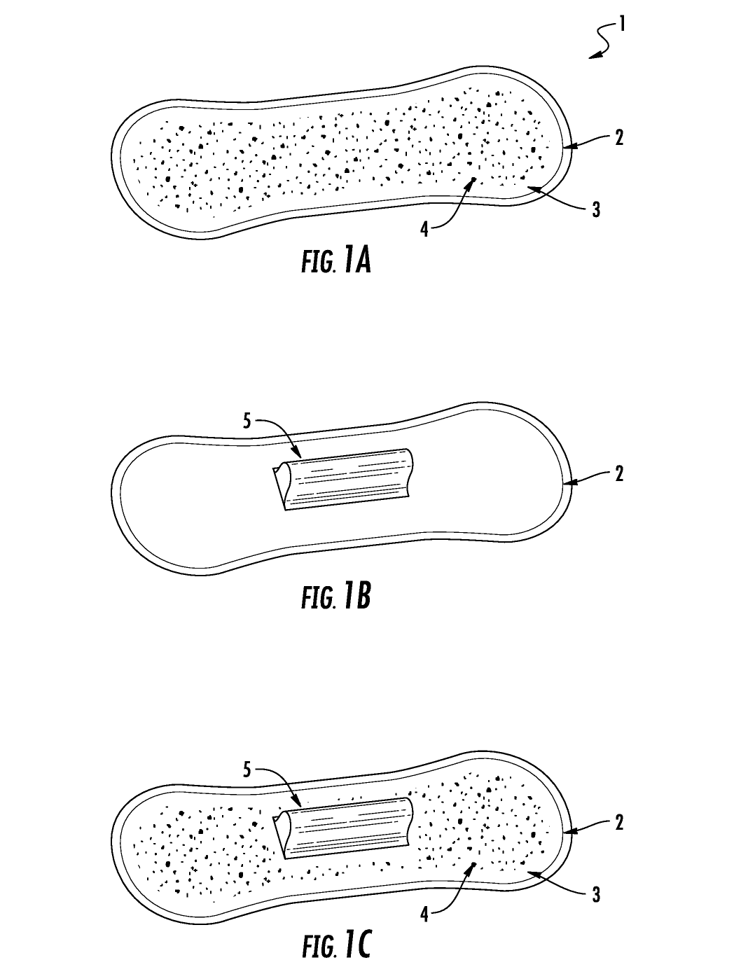

FIG. 1A presents an Enhanced Sanitary Pad having cooling crystals embedded within the absorbent material in an embodiment disclosed herein. An Enhanced Sanitary Pad 1 has a rim 2 that represents two different types of pads; first, it represents a turned over edge for a single plastic backing pad that grips an absorbent material layer using adhesives, heat treatment, glues and or similar modalities; or secondly, it represents a peripheral edge of a two plastic layered sanitary pad where the two plastic layers are attached together through adhesives, heat treatment, glues and or similar modalities such that they together grip the absorbent material layer there between.

A layer of absorbent material 3 including cellulose and polymer is attached to the single plastic layer or between the two layers of plastic. Cooling crystals 4 are mixed into the absorbent material whether combined as cellulose and polymer or within only one or the other or within similar materials sandwiched within the plastic layer(s) or attached thereto using adhesives, heat treatment glues and or similar modalities.

FIG. 1B presents an Enhanced Sanitary Pad having a raised protrusion in an embodiment disclosed herein. A raised protrusion 5 is integrally formed of absorbent material packed together in a tightly formed shape and attached together through adhesives, glue, heat treatment, staples but is preferably made from simply packing the device tightly and inserting the loose end within a suitable opening in the protrusion 5. This raised protrusion 5 is shown at the center of the pad and is formed integrally with the absorbent material 3 so that the raised protrusion can be placed up against the vaginal opening but not within it. The protrusion 5 is meant to capture the menstrual fluids and other liquids that are expelled from the feminine parts and to provide a snug fit thereby avoiding dripping down the inner sides of the users legs.

FIG. 1C presents an Enhanced Sanitary Pad having a raised protrusion 5 and cooling crystals 4 embedded within the absorbent material 3 in an embodiment disclosed herein.

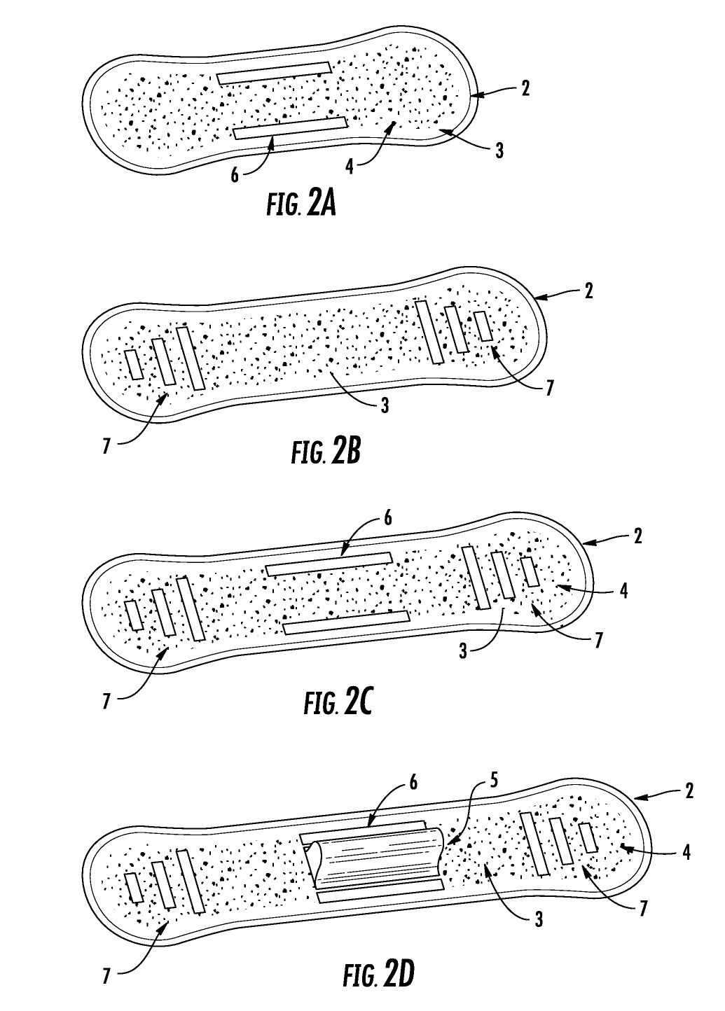

FIG. 2A presents an Enhanced Sanitary Pad having cooling crystals 4 and longitudinal cooling strips 6 in an embodiment disclosed herein. The cooling strips 6 are plastic strips attached to the top surface of the absorbent material 3 that are located midway between the length of the pad and near the top and bottom edge thereof as shown in the figure. The cooling strips 6 have any of the Cooling Gel substances spread thereon such as methyl salicylate topical.

FIG. 2B presents an Enhanced Sanitary Pad having cooling crystals 4 in absorbent material 3 and transverse cooling strips 7 at opposite narrow ends of the pad in an embodiment disclosed herein. The cooling strips 7 decline in size as they approach each opposite end thereof. However, it should be understood that this is only an optional design size for cosmetic reasons and that the sizes do not need to decline but are alternatively the same size or decline inwardly towards the center of the pad. These strips 7 are made from plastic and are similarly attached to the absorbent material layer 3 as were strips 6.

FIG. 2C presents an Enhanced Sanitary Pad having cooling crystals 4 and transverse cooling strips 7 at opposite narrow ends of the pad as well as longitudinal cooling strips 6 in an embodiment disclosed herein.

FIG. 2D presents an Enhanced Sanitary Pad having cooling crystals 4 and transverse cooling strips 7 at opposite narrow ends of the pad as well as longitudinal cooling strips 6 and a raised protrusion 5 in an embodiment disclosed herein. Absorbent material 3 has various cooling crystal 4 embedded therein. The longitudinal cooling strips 6 are presented as being on either side of the raised protrusion 6 and attached to the top surface of the pad.

FIG. 2E presents an Enhanced Sanitary Pad having cooling crystals 4 as well as longitudinal cooling strips 7 disposed on opposite sides of a raised protrusion 5 in an embodiment disclosed herein. Here they are disclosed at top and bottom sides of the raised protrusion 5 or near the outer opposite longitudinal edges of the pad.

FIG. 2F presents an Enhanced Sanitary Pad having cooling crystals 4 embedded in absorbent material 3 as well as transverse cooling strips 7 disposed on opposite ends of the pad 1 as well as having a raised protrusion 5 in an embodiment disclosed herein. The raised protrusion is shown being midway from either narrow end of the pad.

FIG. 3 presents a plastic strip 6, 7, 8 integral with an Enhanced Sanitary pad having a cooling gel 6B, 7B , 8B thereon covered by a removable plastic strip 6A, 7A ,8A used in an Enhanced Sanitary Pad in an embodiment disclosed herein; the dotted line represents the edge of the pad. The cooling strip 6, 7, 8 are shown in other figures herein disposed at various locations. It should be understood that this strip is alternatively attached (adhesive, glue, heat treatment, integrally formed therefrom) to the absorbent material layer 3, or directly to the underlying plastic forming the primary backing layer through a hole (not shown) in the absorbent material layer 3. A cooling gel 6B, 7B , 8B is applied during manufacture and assembly to the surface of the cooling strip 6, 7, 8. Finally, in order for the cooling gel to be available to a user when needed, an optional cover is applied thereto in the form of a removable plastic strip 6A, 7A ,8A having either surface tension adhesion with the gel or a weak adhesive suitable for this purpose.

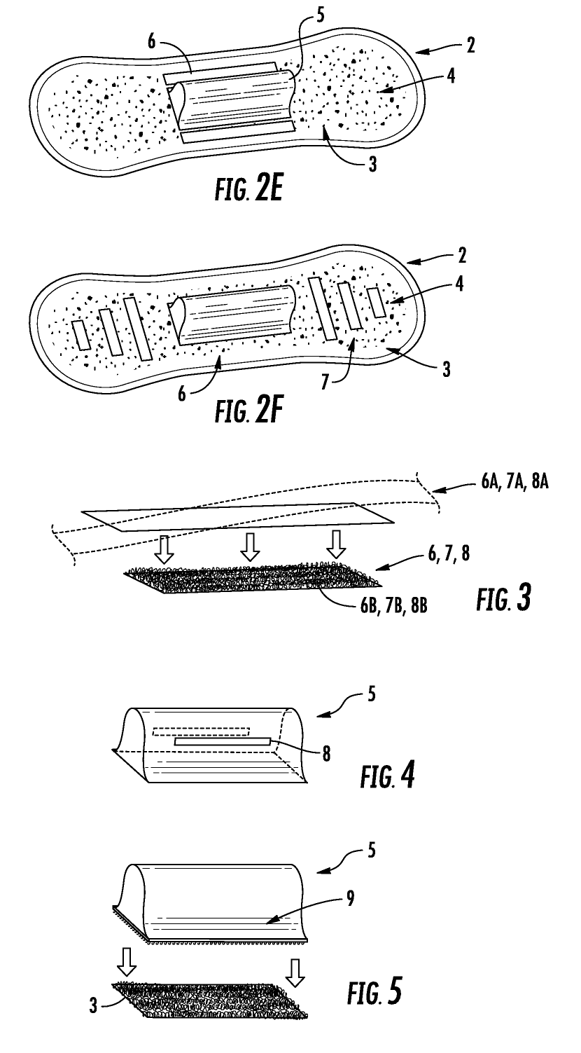

FIG. 4 presents a raised protrusion 5 having a cooling strip 8 thereon for use in an Enhanced Sanitary Pad in an embodiment disclosed herein. The cooling strip 8 is disposed upon a longitudinal side of the protrusion 5 through the use of glues adhesive or heat treatment; of course, it could just as easily be disposed on a transverse side. Additionally, the shape of the protrusion 5 is in any convenient shape so desired. Examples of these shapes include a wedge, a plug, a mushroom, a nipple, rectangular, pyramidal, triangular, circular, elliptical and so forth.

FIG. 5 presents a removable raised protrusion 5 useable in an Enhanced Sanitary Pad in an embodiment disclosed herein. The protrusion 5 is essentially a construct of absorbent material that is formed from one or more pieces of absorbent material; these are brought together then woven together, stitched together, adhesively connected, glued, heat treated or chemically treated to maintain an appropriate physical size. An alternative manufacturing option would be to have the material wound about a supporting jig in association with a rotating dispensing spool.

The supporting jig is centrally disposed and rotates on its axis while the spool similarly rotates on its axis thereby forming the protrusion; alternatively, the jig is stationary and the spool of material rotates on its axis and also revolves about the supporting jig using an armature for this purpose. The various motions described are provided by electric motors disposed thereabout. In either scenario, the material is then cut and the end thereof is attached to the rest of the absorbent material forming the protrusion by physical insertion of its end into a opening in the absorbent material and wrapping it together or through the use of glue, adhesives, heat treatment or chemical bonding agents.

A strip 9 of velcro material is applied to the bottom of the protrusion 5 for selective engagement with a corresponding piece of velcro strip similarly attached to a top portion of absorbent material of the pad. Alternatively, the corresponding piece of velcro material is attached to an outer first or second top or bottom layer of plastic backing material forming the pad. A flap (not shown) of absorbent material (or plastic backing material) formed from the absorbent material (or plastic backing material) integrally attached to the pad and plastic backing layers also has an optional velcro material strip attached thereto. This flap strip of velcro attaches to the first one integral with the pad so as to ensure that when the protrusion is removed a user can still use the pad without discomfort turning the flap over onto the integrally formed velcro material attached to a top portion of absorbent material or onto an outer first or second top or bottom layer of plastic backing material.

Another option instead of velcro is to have a double sided strip of material 9 permanently attached on one side thereof using strong adhesives or glues to the bottom of the protrusion 5. The other side of the strip of material 9 has another adhesive, glue or sticky gel on its underside that is to attach to a user clothing. Thus, a user can place this underside in the undergarment and attach it thereto so as to maintain its position on the undergarment using appropriately strong adhesives, glues, sticky gels or similar modalities. Thus, a user can remove the protrusion 5 from the pad and use it separately from the pad as desired or it can be bought separately at a store. Another option has a removable plastic strip attaching to this other side of the strip of material 9 for protection of the attachment modality, namely, glues, adhesive or sticky gels.

It should be apparent that in the event the protrusion is not removable then it either its underside is attached using heat treatment, glues, adhesives or chemical additives to the absorbent material formed on the plastic backing layers.

The above-described embodiments are merely exemplary illustrations of implementations set forth for a clear understanding of the principles of the invention. Many variations, combinations, modifications or equivalents may be substituted for elements thereof without departing from the scope of the invention. Therefore, it is intended that the invention not be limited to the particular embodiments disclosed as the best mode contemplated for carrying out this invention, but that the invention will include all the embodiments falling within the scope of the appended claims.

What is claimed is:

1. A sanitary pad comprising:
a first plastic layer having
an absorbent material attached thereto and
a protrusion attached to the sanitary pad and extending out therefrom.

2. The sanitary pad of claim 1, further comprising:
cooling crystals embedded within the absorbent material.

3. The sanitary pad of claim 1, further comprising:
a cooling strip attached to the sanitary pad.

4. The sanitary pad of claim 3, wherein the cooling strip is disposed on a side of the protrusion.

5. The sanitary pad of claim 3, wherein the cooling strip is disposed adjacent to a side of the protrusion.

6. The sanitary pad of claim 3, wherein the cooling strip is disposed at an end of the sanitary pad.

7. The sanitary pad of claim 1, wherein the protrusion is removable from the sanitary pad.

8. The sanitary pad of claim 7, wherein the protrusion has a first attachment integrally attached thereto.

9. The sanitary pad of claim 8, wherein the sanitary pad has a corresponding second attachment integrally attached thereto for removal of the protrusion.

10. A hygiene warning system comprising:
a first plastic sheet having
an absorbent layer attached thereto and
cooling crystals associated with the absorbent layer.

11. The hygiene warning system of claim 10, further comprising:
a cooling strip attached to the hygiene warning system.

12. The hygiene warning system of claim 11, wherein the cooling strip is disposed on a side of a protrusion associated with the hygiene warning system.

13. The hygiene warning system of claim 11, wherein the cooling strip is disposed adjacent to a side of a protrusion associated with the hygiene warning system.

14. The hygiene warning system of claim 11, wherein the cooling strip is disposed at an end of the hygiene warning system.

15. The hygiene warning system of claim 10, further comprising:
a protrusion associated with the hygiene warning system.

16. The hygiene warning system of claim 15, wherein the protrusion is removable from the sanitary pad.

17. The hygiene warning system of claim 16, wherein the protrusion has a first attachment integrally associated with it.

18. The hygiene warning system of claim 17, wherein the hygiene warning system has a corresponding second attachment integrally attached thereto for removal of the protrusion.

19. A liquid absorbing napkin comprising:
a base layer having
an absorbent material integrally attached to the base layer;
wherein the liquid absorbing napkin has a cooling system associated therewith.

20. The liquid absorbing napkin of claim 19, wherein the cooling system is from a group of cooling systems comprising: a cooling strip, cooling crystals.
